(19) United States
(12) Patent Application Publication
(10) Pub. No.: US 2020/0069223 A1
(43) Pub. Date: Mar. 5, 2020

(54) SYSTEM FOR DETECTION OF FALLS

(71) Applicant: Connectus Life Pty Ltd, Clayfiedl, Queensland (AU)

(72) Inventors: Andrew Steptoe, Clayfield, Queensland (AU); Benjamin Slater, Albany Creek, Queensland (AU)

(21) Appl. No.: 16/492,886

(22) PCT Filed: Apr. 28, 2017
(86) PCT No.: PCT/AU2017/050385

§ 371 Date: Sep. 10, 2019

(30) Foreign Application Priority Data
Mar. 14, 2017 (AU) 2017900883

(51) Int. Cl. A61B 5/11; A61B 5/00; G16H 40/67; G16H 80/00

(52) U.S. Cl. CPC A61B 5/1117; A61B 5/681; A61B 5/0022; A61B 2562/0219; G16H 40/67; G16H 80/00; A61B 5/747

(57) ABSTRACT

The invention resides in a system to assist in the care and independence of at-risk persons, comprising a first smart device operated by a carer of an at-risk person, and a second smart device in communication with the first generic smart device, being a wearable smart device worn by the at-risk person and comprising at least an internal accelerometer and GPS, whereupon detection of a fall by the at-risk person by the internal accelerometer the second smart device determines its location through operation of the internal GPS and transmits its location to the first smart device.

### PRIORITY

The present specification draws priority from AU 2017900883, “System for Detection of Falls”. It is also intended that the disclosure in Australian Provisional Application No. 2017900883 be incorporated by reference in this and any national application for Letters Patent.

### TECHNICAL FIELD

The present invention relates to the field of providing independence and preserving the dignity of elderly, disabled and other at-risk people. The invention, in particular, is directed to the provision of assistance in a discreet manner, and to ensure the peace-of-mind of caretakers or carers, and that of their charges. Specifically the invention is designed to preserve the dignity of the latter when out in public places.

### BACKGROUND

Prior means of providing independence to at-risk persons, such as the elderly, comprise systems including devices such as the ‘alert button’, which provides a means for those wearing the device to call for help in the event of an accident. This, however, relies on the at-risk person being conscious for long enough to activate the alert button. Unfortunately, in the event of a heart attack, stroke or accident that otherwise renders the person unconscious, the system breaks down and does not work.

There are certain automated systems that monitor remotely those at risk for signs of potential danger, typically by monitoring a person's posture to determine if they have fallen over, as an indication of an accident or incident having occurred. These systems tend to be confined to the person's abode, and utilise visual or infra-red monitoring cameras and other sensors, wherein the person not only loses personal privacy, but is also deprived of the ability to independently leave their home.

Other personal devices which detect the acceleration of a fall to trigger an alarm, are typically connected to the person's landline phone and so will only operate within a certain distance of their premises. This suffers from the further setback of modern telecommunication systems gradually phasing out the domestic landline based telephone.

Other prior art devices require a specific piece of hardware to be purchased and tend to be bulky, unsightly and otherwise inconvenient for the user to carry around. Not uncommonly, for obvious reasons, they also draw public attention to the user. These limitations make it far more likely that such devices will be neglected by those in need of them, and by their non-use, place themselves at great risk.

Finally, existing algorithmic means for detection of falls are rudimentary at best. No fully-portable algorithmic means of detecting a fall that can provide the level of sensitivity necessary for at-risk persons is available; existing algorithmic means rely on very large or very sudden changes for use in industrial environments, and so cannot be adequately adapted to suit the needs of at-risk persons in everyday situations.

It would therefore be advantageous to provide a system where an at-risk person may possess a substantial degree of independence, autonomy, dignity and freedom of movement without sacrificing the ability of carers or caretakers to remotely monitor their wellbeing. It is also particularly advantageous that the alert device is not one that indicates to the public at large that the at-risk person is being monitored, but resembles an ordinary device which may also be worn by a non-disabled person.

### STATEMENTS OF THE INVENTION

In one aspect, the invention resides in a system to assist in the care and independence of at-risk persons, comprising a first smart device operated by a carer of an at-risk person, and a second smart device in communication with the first smart device, being a wearable smart device worn by the at-risk person which includes an accelerometer and GPS, whereupon detection of the at-risk person falling by the accelerometer, the second smart device transmits an alert signal, including location determined through operation of the GPS, to the first smart device.

In an embodiment, the second smart device further includes a microphone adapted so that upon detection of the fall by the accelerometer, sounds in the vicinity of the second smart device are recorded and transmitted to the first smart device.

In an embodiment, at any time when the two smart devices are in communication with one another, and independent of the accelerometer, the first smart device is able to request the location of the second smart device.

In an embodiment, the second smart device includes an internal battery and means of monitoring the battery status, including percentage of battery charge, wherein battery status information is transmitted to and can be displayed by the first smart device. In an embodiment, the battery status information is transmitted to the first smart device at regular time intervals. In an embodiment, the battery status information is transmitted to the first smart device at predefined percentage charge levels selected from one or more of 90%, 80%, 75%, 60%, 50%, 40%, 30%, 25%, 10%, 5%, 3%, 2% and 1% of remaining charge. In an embodiment, the transmission of the battery status information to the first smart device is due to an independent request issued by the first smart device.

In an embodiment, the second smart device is capable of transmitting a message comprising the second smart device's location detected through the GPS at the request of either the carer or the at-risk person.

In an embodiment, prior to transmitting the alert signal to the first smart device, the at-risk person is able to notify the second smart device that a fall was falsely indicated, thereby cancelling the alert signal to the first smart device, and failure to receive the notification within a predefined interval of time will continue to validate the alert signal.

In an embodiment, the accelerometer employs at least one algorithm to determine if an act by the at-risk person is a real fall; the algorithm able to be adjusted in order to alter fall detection sensitivity, thereby allowing for the second smart device to exclude certain acts which could result in a false indication and initiating the transmission of an invalid alert signal.

In an embodiment, the first smart device is any smart device including a smart watch, cellular phone or tablet computer. In an embodiment, the second smart device is any smart device including a smart watch, cellular phone or tablet computer.

In an embodiment, a second smart device is able to be in communication with a plurality of first smart devices. In an embodiment, a first smart device is able to be in communication with a plurality of second smart devices.

In an embodiment, the second smart device comprises a pair of smart devices in communication with one another, including a primary monitoring device adapted to detect a fall by the at-risk person, and an intermediary communication device adapted to facilitate communications between the primary monitoring device and the first smart device.

A second aspect of the invention resides in a method in assisting in the care and independence of at-risk persons, including the steps of:
operating a first smart device by a carer of the at-risk person;
operating a second smart device by the at-risk person;
initiating wireless communication between the first and second smart devices;
wherein upon detection of a fall by an accelerometer of the second smart device, determining the location of the second smart device through a GPS of the second smart device; and
transmitting an alert signal including location of the second smart device to the first smart device.

In an embodiment, upon detection of a fall by the accelerometer, the second smart device activates an internal microphone; the microphone records audio local to the second smart device; and transmits the recorded local audio, together with the location of the second smart device, as a part of the alert signal to the first smart device.

In an embodiment, the method may be coordinated via a computer system and wireless communications protocol which runs a computer program including at least one instruction, which on execution, performs one or more of the method steps. In an embodiment, the method utilises a data signal encoding at least one computer readable instruction, which on execution, performs one or more of the method steps.

In an embodiment of the method, the method is implemented using an embodiment of the system of the invention.

### BRIEF DESCRIPTION OF THE DRAWINGS

In order that the present invention be better understood and put into practice, reference is made to the accompanying drawings wherein:

FIGS. 1A, 1B & 1C show three embodiments of the system, detailing the communicative relationship between the smart devices;

FIG. 2 shows a plurality of functions offered by the second smart device in various embodiments;

FIG. 3 shows the basic method of the invention and the method implemented by certain embodiments of the system;

FIG. 4 shows an expanded method of the invention requiring user input for verification, as well as certain embodiments of the method that may be implemented by certain embodiments of the system of the invention; and

FIG. 5 shows a network of at-risk persons and carers, showcasing how a plurality of carers and at-risk persons may interconnect.

### DETAILED DESCRIPTION OF PREFERRED EMBODIMENTS

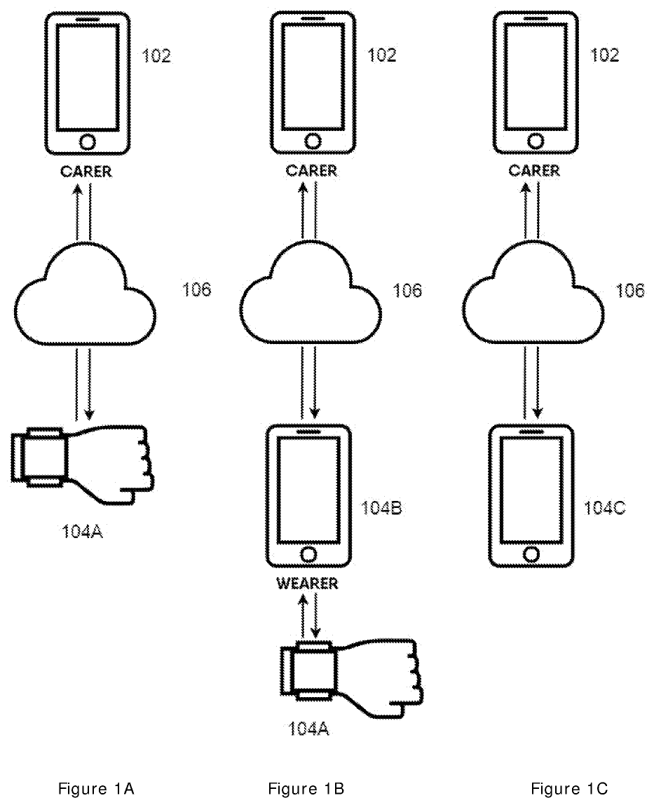

With reference to FIGS. 1A-1C and in a first aspect, the invention is a system comprising a first smart device 102 and a second smart device 104, wherein the first smart device 102 is operated by a carer for an at-risk person and the second smart device 104 is a wearable smart device 104A worn by the at-risk person and comprises at least an internal accelerometer and GPS, and the two smart devices are in communication with one another through communication means 106.

In certain embodiments and as shown in FIG. 1A, the second smart device 104 is a wearable smart device 104A that is worn by the at-risk person. In this embodiment, the wearable smart device 104A is in direct communication with the first smart device 102.

In certain embodiments as shown in FIG. 1B, the second smart device 104 may comprise a wearable smart device 104A and an intermediary communication device 104B, both of which are operated by the at-risk person. The wearable smart device 104A acts as a primary monitoring device, in that it serves to monitor the at-risk person and detect a fall. The intermediary communication device may be a smart phone, mobile phone, tablet computer or other form of portable smart device capable of communications. In this embodiment, the wearable smart device 104A may communicate with the first smart device 102 by relaying the alert and any accompanying data through the intermediary communication device 104B. This embodiment is particularly applicable when the wearable smart device 104A is incapable of direct communications with the first smart device 102.

In certain alternative embodiments as shown in FIG. 10, the second smart device may be any generic smart device 104C that is adapted to be carried by the at-risk person.

Where reference is made in this specification to the second smart device 104 communicating with the first smart device 102, it is within the scope of the invention for this to be interpreted to include the wearable smart device 104A, a wearable smart device 104A paired with an intermediary communication device 104B, and a carryable smart device 104C.

Although not shown, it is within the scope of this invention for the first smart device 102 operated by the carer to also comprise multiple linked smart devices in communication with one another.

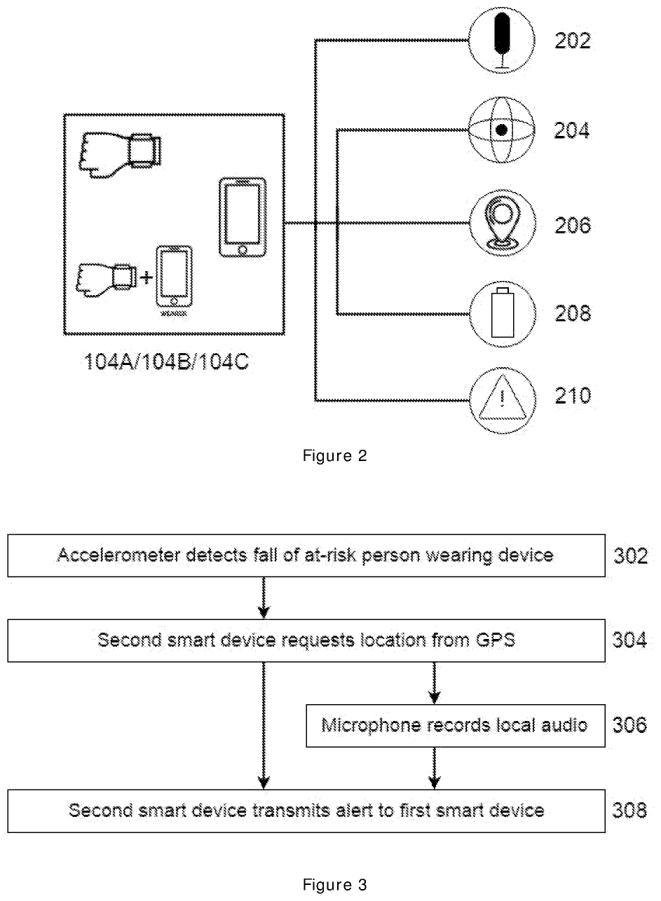

FIG. 2 depicts a range of technical features within a range of embodiments of the system. Not all technical features shown in FIG. 2 are regarded as essential. In an embodiment, the second smart device 104 is operated by an at-risk person and receives input from the second smart device's inbuilt GPS 206 and accelerometer 204. In an embodiment, an incident or accident resulting in the at-risk person falling may be detected by the accelerometer 204. This triggers the transmission of an alert signal to the first smart device 102, comprising at least the location of the second smart device as determined by the inbuilt GPS 206. This may allow for a message to be transmitted by the second smart device 104 to the first smart device alerting the carer to the incident.

The accelerometer 204 may determine a fall through the execution of one or more algorithms that receive information from the physical components of the accelerometer 204 and determine the likelihood of a fall having taken place. In certain embodiments, these algorithms may be able to be calibrated by the carer in order to account for different requirements, thereby adjusting the sensitivity of the system to detecting a fall and initiating an alert. For example, the algorithms may require adjustment to account for the shaking of an at-risk person with Parkinson's Disease or similar, wherein shaking may trigger a false alarm.

In an embodiment, the second smart device further comprises a microphone 202 and the alert may further comprise a sound transmission, which may provide valuable information on the state of the at-risk person. In this embodiment, upon detection of a fall the second smart device 104 may activate an inbuilt microphone 202. The microphone 202 may then receive and record sounds in the vicinity of the second smart device 104 which are then able to be transmitted to the first smart device 102. This transmission may be an ongoing transmission, or may comprise a discrete audio file comprising a recording. In this embodiment, the audio may alert the carer to if the at-risk person is conscious or unconscious due to the presence or absence of particular sounds.

In some embodiments wherein the second smart device 104 is a wearable smart device 104A used in conjunction with an intermediary communication device 104B, the microphone 202 may be within the intermediary communication device 104B. In these embodiments, upon detection of a fall by the accelerometer 204, a signal may be sent to the intermediary communication device 104B to activate the device's internal microphone 202. In some embodiments, there may be a microphone 202 utilised for the invention within both the wearable smart device 104A and the intermediary communication device 104B.

This system provides a means for the carer to be rapidly alerted to an accident or incident, even in the event of said incident or accident rendering the at-risk person unconscious or otherwise unable to manually request help. This is advantageous in that it eliminates a potential flaw in many prior art systems which require manual transmission of an alert or alarm signal. As accidents resulting in unconsciousness have a strong potential to be life-threatening—particularly for a person who is already at risk of health problems—by automating the alert process, the survivability of the at-risk person may be improved by potentially improving the response time of the carer. The technical features of various embodiments may offer further peace of mind to the carer as more detailed information may be provided to them.

A key feature of the invention is that the system is automatically triggered by the at-risk person falling over, collapsing, fainting or otherwise experiencing a falling event. Such a situation is regarded as indicative of an accident and is more common in certain at-risk sections of the population, such as elderly people, neurodegenerative disease sufferers and other population groups. A falling event may culminate in a loss of consciousness, or alternatively may be triggered by events such as fits, spasms or exhaustion, or may be environmentally triggered such as the at-risk person tripping. Many of these may result in, or be precipitated by, a loss of consciousness, and so it is advantageous to have an automated system that does not rely upon the at-risk person to trigger the alert.

This advantageous feature is detailed the flow diagram of FIG. 3, wherein it is shown that detection of an at-risk person falling 302 triggers the system to immediately request the location of the second smart device from the GPS 304. This data is then transmitted as an alert to the first smart device in the ‘transmission’ stage 308. In an embodiment, there may be the additional step of activating the microphone in order to record local audio 306, which is transmitted to the first smart device as part of the alert during the transmission stage 308.

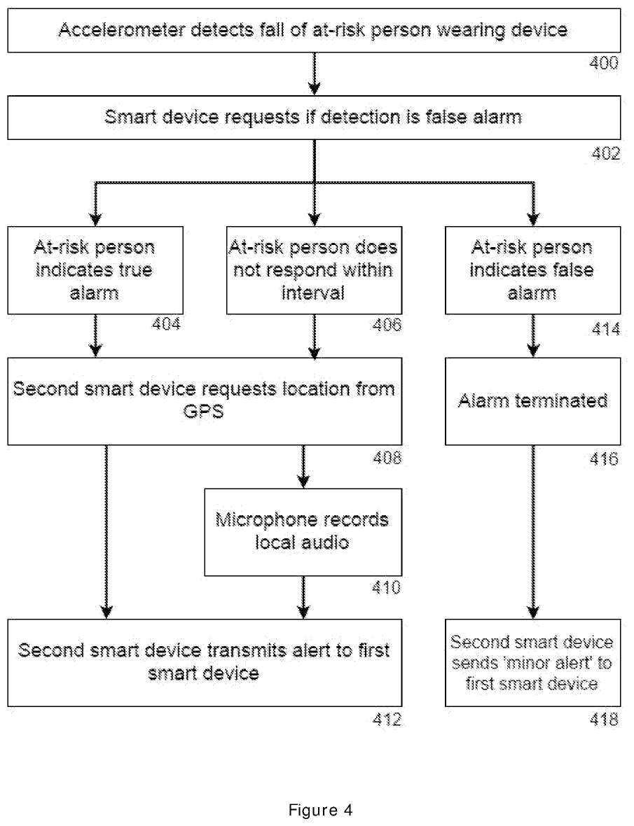

In an embodiment depicted in FIG. 4, following a perceived incident, the second smart device may not immediately transmit an ‘alert’ to the first smart device. As with the embodiment shown in FIG. 3, the ‘detection’ stage 400 involves the accelerometer of the second smart device detecting a fall. However, in this embodiment, the second smart device may provide for a short window 402 in which the at-risk person may provide input that prevents the alert from being transmitted. If the at-risk person responds confirming that there was an incident 404 or fails to respond within a certain timeframe 406, the system progresses as standard; the second smart device's location is determined 408 and transmitted 412. As before, there may be embodiments wherein the microphone of the second smart device activates and records 410.

However, the at-risk person may also indicate that the accident is a false alarm 414. This terminates the alarm state 416, and may prevent the second smart device from transmitting the message to the first smart device. Alternatively, upon termination of the alarm state the second smart device may send a ‘minor’ alert message 418 to the first smart device, comprising a short message indicating that there was a perceived incident that was cancelled by the at-risk person. In an embodiment, the alert may be cancelled by the at-risk person even after it has already been transmitted to the first smart device.

In an embodiment, the current battery status 208 of the second smart device 104 is transmitted to the first smart device. This may be transmitted at set intervals, for example every half-hour, every hour, every six hours, or in alternative arrangements such as at set times each day. In an alternative embodiment, the current status 208 is transmitted continually, allowing for ‘live’ monitoring. In an alternative embodiment, the current status 208 is transmitted upon request from the first smart device 102. In a further alternative embodiment, the current status 208 of the battery of the second smart device is transmitted at intervals dependent upon the status 208 of the battery. For example, the current status 208 of the battery may be transmitted to the first smart device at one or more of 90%, 80%, 75%, 60%, 50%, 40%, 30%, 25%, 10% or 5% of remaining charge. There may be additional ‘critical’ transmissions, for example if a fault is detected within the battery, or the battery charge drops below 5%, 3%, 2% or 1%. Alternative battery status 208 transmissions may additionally be transmitted.

In an embodiment of the invention, the first smart device 102 may be able to contact the second smart device 104 and request location data from the GPS 206 of the second smart device 104. In an embodiment, the respective smart devices may be adapted to speed-dial the other smart device and establish communication. In an embodiment of the invention, the second smart device 104A may provide a means for the at-risk person to manually trigger an alert 210. The manual alert 210 permits an at-risk person to request help in the absence of a fall and may be for a different medical reason or any one of a range of scenarios wherein the at-risk person may want or need assistance.

In an embodiment of the invention, independence of an at-risk person may be promoted by permitting them to move and/or travel to a wide variety of locations without their device being out of range of transmitting an alert message. In at least one embodiment, the first and second smart devices 102, 104 communicate through conventional communication networks, which may utilise generic SIM cards. In this embodiment, the at-risk person wearing the second smart device 104 may be able to travel to any location where a communication network signal may be detectable and therefore may still be able to be remotely monitored for the occurrence of an accident or incident. The second smart device 104 may in certain embodiments be a wearable smart device 104A combined with the intermediary communication device 104B in order to provide full communication with the first smart device 102. This may therefore promote independence of the at-risk person by reducing their dependency upon full-time carers, while potentially providing peace of mind to the carer by potentially ensuring that they may be rapidly notified of the at-risk person being involved in an incident or accident.

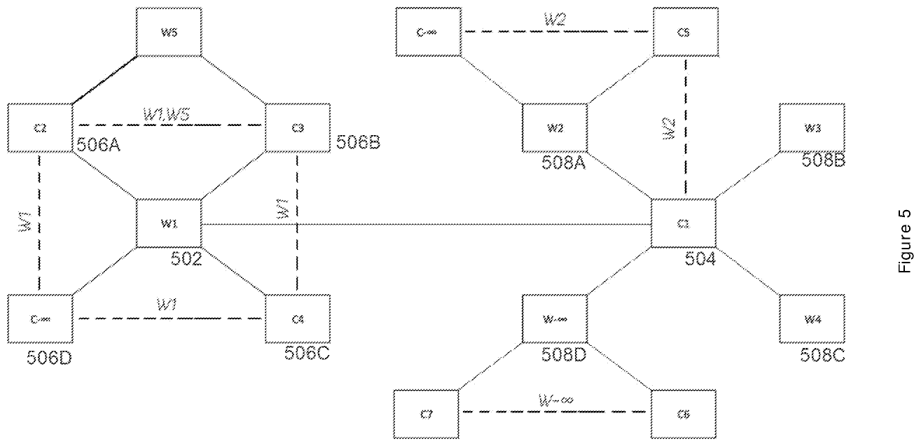

In an embodiment shown in FIG. 5, the system may comprise a network of first and second smart devices interconnected in a range of manners. For example, a single at-risk person's second smart device 502 may be monitored by multiple first smart devices 504, 506A-D. Alternatively, a single carer may, through a single first smart device 504, monitor a number of second smart devices 502, 508A-D. In such embodiments, signals sent between smart devices will additionally carry identification markers in order to indicate the device of origin.

In certain embodiments of the system of the invention, such a system may rely on custom software packages designed to operate on each smart device, being a ‘monitoring software package’ on the first smart device and a ‘detection software package’ on the second smart device. The detection software package may provide for a means by which the second smart device's location is requested from the inbuilt GPS and packaged with an ‘alert’ message that is transmitted to the monitoring software package of the first smart device. The detection software package may further provide for a means by which the various other aspects of the invention may be monitored, and information about which may be transmitted to the first smart device. For example, the detection software package may determine the intervals at which the status of the second smart device's battery is transmitted to the first smart device. In an embodiment, the detection software package may also provide a means for ‘packaging’ the data of an alert, comprising one or more of the accident details, the location data, the sound and any other potentially valuable pieces of information. This alert package may then be transmitted to the first smart device, whereupon the package may be ‘unpacked’ by the monitoring software package and thus displayed for the carer.

In certain embodiments, the two software packages provide a means for the carer and at-risk person to easily get in contact with one another. This may comprise a speed-dial system that interfaces with a telephone component within the respective smart devices. It may comprise other means of establishing communications between the at-risk person and carer, for example through a means that does not utilise the inbuilt telephone system of the smart devices. This may be relevant in the event wherein one or more of the smart devices is not intended to serve as a mobile telephone and so lacks the necessary software. The communication means may be audio, visual or a combination thereof.

Other aspects of the broad inventive concept relate to a corresponding method, computer program and data signal. The method facilitates remote monitoring of an at-risk person by a carer through the communication of a first and second smart device, wherein the second smart device is a wearable smart device worn by the at-risk person and detects if they fall.

In an aspect of the invention, there is provided a method by which a carer of an at-risk person may be alerted to an incident, accident or other difficulty experienced by the at-risk person. The method is carried out by a first and a second smart device, operated by the carer and at-risk person respectively. The method of an embodiment of the invention utilises the following steps as shown in FIG. 3: The at-risk person, who is carrying or wearing the second smart device, falls over; the rapid change in direction is detected by an accelerometer built into the second smart device 302; and the second smart device determines that a fall has taken place, requests the location of the second smart device 304 and sends an alert message 308 to the first smart device, whereupon it is displayed either audibly and/or visibly.

The importance of the method being automatically activated by the event of the at-risk person falling 302 lies in that the system does not rely upon the at-risk person maintaining consciousness or motor function for any period of time. As with the system of the invention, reliance upon the consciousness and motor function of the at-risk person in order to alert the carer to a falling event may be detrimental to the at-risk person's health. Should they have collapsed due to, for example, an epileptic fit, they would be unable to trigger any form of alert until the fit had subsided and they regained motor function. Should the at-risk person have experienced a loss of motor control or consciousness for another medical condition (or age), they would be similarly unable to request help.

As with the system of the invention, certain embodiments of the method of the invention may include the additional step of the microphone activating and recording local audio 306. This local audio may then be transmitted to the first smart device in the alert transmission stage 308. The recorded audio may be a set interval of recording, or may be an ongoing transmission. In some embodiments wherein the second smart device is used in conjunction with an intermediary communication device, the microphone may be within the intermediary communication device. In these embodiments, upon detection of a fall by the accelerometer 302, a signal may be sent to the intermediary communication device to activate the device's internal microphone in order to record local audio 306. In some embodiments, recording of local audio 306 may be performed by microphones in both the second smart device and the intermediary communication device.

A further embodiment of the method of the invention is shown in FIG. 4, wherein the ‘fall detection’ stage 400 is once again the initiating stage. This is followed by the ‘Confirmation Request’ stage 402 wherein for a short interval of time, the smart device 104 inquires if the fall detection was a false positive and there is no alarm. A ‘False Alarm’ indication 414 results in ‘Alarm Termination’ 416, while a ‘Failure to Respond’ 406 will trigger the remainder of the method as previously described. In short, a failure to respond 406 triggers the ‘Location determination’ stage 408, followed by the ‘alarm transmission’ stage 412, wherein the carer is alerted to the incident as well as the location of the second smart device 104A.

In certain embodiments, the ‘Confirmation Request’ stage 402 may allow for the at-risk person to confirm the accident 404, which as with ‘Failure to Respond’ 406 will trigger the remainder of the method. The ‘Confirmation Request’ stage 402 allows for the method to be actioned without waiting for an interval of time to expire.

In certain embodiments, the method may include the additional step of the microphone activating and recording local audio 410. This local audio may then be transmitted to the first smart device in the alert transmission stage 412. The recorded audio may be a set interval of recording, or may be an ongoing transmission.

In certain embodiments, termination of the alarm via the ‘Alarm Termination’ stage 416 may trigger a ‘minor alert’ 418, notifying the carer of the false alarm. This is to allow for the carer of the at-risk person to be kept abreast of the situation, and if it becomes apparent that a large number of false alarms are being triggered, the carer or at-risk person may initiate a recalibration of the algorithms computing the input from the accelerometer within the second smart device.

Throughout this specification, unless the context requires otherwise, the word “comprise” or variations such as “comprises” or “comprising”, will be understood to imply the inclusion of a stated integer or group of integers but not the exclusion of any other integer or group of integers.

Those skilled in the art will appreciate that the invention described herein is susceptible to variations and modifications other than those specifically described. The invention includes all such variation and modifications. The invention also includes all of the steps, features, components and/or devices referred to or indicated in the specification, individually or collectively and any and all combinations or any two or more of the steps or features.

Other definitions for selected terms used herein may be found within the detailed description of the invention and apply throughout. Unless otherwise defined, all other scientific and technical terms used herein have the same meaning as commonly understood to one of ordinary skill in the art to which the invention belongs.

Where reference is made to communication standards, methods and/or systems, robots or devices may transmit and receive data via a variety of forms: 3G, 4G, 5G, (CDMA/GSM), Wi-Fi, Bluetooth, other radio frequency, optical, acoustic, magnetic, GPS/GPRS, or any other form or method of communication that may become available from time to time.

In the preceding description, where a feature or device such as “processor”, “smart device”, etc., is described with reference to separate figures and different reference numerals, it will be understood that each different reference numeral refers to a different embodiment of the feature or device, but that the function of the feature or device remains analogous, even though the interworking relationship between the features may be different in different embodiments.

1. A system to assist in the care and independence of at-risk persons, comprising:
a first smart device operated by a carer of an at-risk person; and
a second smart device in communication with the first smart device, being a smart device operated by the at-risk person which includes an accelerometer and GPS;
whereupon detection of the at-risk person falling by the accelerometer, the second smart device transmits an alert signal, including location determined through operation of the GPS, to the first smart device.

2. The system of claim 1 wherein the second smart device further includes a microphone adapted so that upon detection of the fall by the accelerometer, sounds in the vicinity of the second smart device are recorded and transmitted to the first smart device.

3. The system of claim 1 wherein at any time when the two smart devices are in communication with one another, and independent of the accelerometer, the first smart device is able to request the location of the second smart device.

4. The system of claim 1 wherein the second smart device includes an internal battery and means of monitoring the battery status, including percentage of battery charge, wherein battery status information is transmitted to and can be displayed by the first smart device.

5. The system of claim 4 wherein the battery status information is transmitted to the first smart device at regular time intervals.

6. The system of claim 4 wherein the battery status information is transmitted to the first smart device at predefined percentage charge levels selected from one or more of 90%, 80%, 75%, 60%, 50%, 40%, 30%, 25%, 10%, 5%, 3%, 2% and 1% of remaining charge.

7. The system of claim 4 wherein the transmission of the battery status information to the first smart device is due to an independent request issued by the first smart device.

8. The system of claim 1 wherein the second smart device is capable of transmitting a message comprising the second smart device's location detected through the GPS at the request of either the carer or the at-risk person.

9. The system of claim 1 wherein, prior to transmitting the alert signal to the first smart device, the at-risk person is able to notify the second smart device that a fall was falsely indicated, thereby cancelling the alert signal to the first smart device; and
failure to receive the notification within a predefined interval of time will continue to validate the alert signal.

10. The system of claim 1 wherein the accelerometer employs at least one algorithm to determine if an act by the at-risk person is a real fall; the algorithm able to be adjusted in order to alter fall detection sensitivity, thereby allowing for the second smart device to exclude certain acts which could result in a false indication and initiating the transmission of an invalid alert signal.

11. The system of claim 1 wherein the first smart device is any smart device including a smart watch, cellular phone, tablet computer and other devices capable of internet connectivity, or any working combination thereof.

12. The system of claim 1 wherein the second smart device is any smart device including a smart watch, cellular phone, tablet computer and other devices capable of internet connectivity, or any working combination thereof.

13. The system of claim 1 wherein the second smart device comprises a pair of smart devices in communication with one another, the pair of smart devices including:
a primary monitoring device adapted to detect a fall by the at-risk person; and
an intermediary communication device adapted to facilitate communications between the primary monitoring device and the first smart device.

14. The system of claim 1 wherein a second smart device is able to be in communication with a plurality of first smart devices.

15. The system of claim 1 wherein a first smart device is able to be in communication with a plurality of second smart devices.

16-20. (canceled)
